(19) United States
(12) Patent Application Publication
(10) Pub. No.: US 2020/0111568 A1
(43) Pub. Date: Apr. 9, 2020

(54) COMPUTER-IMPLEMENTED PATIENT PROXIES AND METHODS OF GENERATING AND PRESENTING PATIENT PROXIES

(71) Applicant: Darroch Medical Solutions, Inc., San Diego, CA (US)

(72) Inventors: Colby Bishop, San Diego, CA (US); Anthony Shao, Hillsborougn, CA (US); Jacques Yeager, Riverside, CA (US)

(21) Appl. No.: 16/594,111

(22) Filed: Oct. 7, 2019

Related U.S. Application Data
(60) Provisional application No. 62/742,475, filed on Oct. 8, 2018.

(51) Int. Cl. G16H 40/63; G16H 70/20; G16H 20/17

(52) U.S. Cl. CPC G16H 40/63; G16H 20/17; G16H 70/20

(57) ABSTRACT

Computer-implemented patient proxies and methods of generating and presenting patient proxies are provided comprising aggregated data relating to a patient outputted by one or more medical instruments, one or more boundary parameters for the data, a data analytics system, a communications system, and an alarm. The data analytics system determines which data comes from a particular medical instrument and whether any data is outside the boundary parameters, and determines whether a patient may experience an adverse event through comparisons between the patient's progression and trends and an adverse event repository. The communications system communicates information about a patient's medical condition based on the data determinations of the data analytics system. The alarm sounds when selected data is outside pre-determined boundary parameters or a particular medical instrument has stopped functioning or changed its rate, or if a patient may experience an adverse event.

### CROSS-REFERENCE TO RELATED APPLICATION

This application claims priority to and benefit of U.S. Patent Application No. 62/742,475, filed Oct. 8, 2018, which is hereby incorporated by reference in its entirety.

### FIELD OF THE DISCLOSURE

The present disclosure relates to patient proxies that provide medical practitioners with bedside treatment and medical device data, patient progression, and patient-specific analytics information in real time.

### BACKGROUND

In the current hospital ecosystem, medical practitioners typically assess the medical status of their patients by aggregating the information available from various devices (e.g. infusion pumps, vital signs monitors, bed scales, etc.) that are connected to the patient or by aggregating the responses of patients to questions posed directly at their bedside, or through a combination of the two. In the current hospital system, remote access of data from devices by practitioners occurs through the hospital Electronic Medical Records (EMR) system. Device data is loaded into the EMR in one of two ways. First, it is entered manually by nurses using workstations located either in the patient's room, or at a nurse station. Second, data is automatically transmitted by devices to medical device vendor-specific servers using the hospital network. Practitioners, with access to the medical data from the various medical instruments, then deduce the state of the patient's health and determine the treatment appropriate for recovery.

At present, practitioners encounter several problems in their determination of patient health and treatment from the information available from medical devices. For instance, data from medical devices (including alarms) is not available to doctors and nurses in a timely (including, as needed, in a real-time) manner. Data transmitted to the EMR is sparse, often loaded to the EMR as a batch process, and available at most a few times a day. As a result, their intended function as a monitoring system is defunct. As another instance, device data have to be integrated by practitioners themselves based on their knowledge and experience to derive patient health information. For example, a simultaneous rise in temperature and fall in blood oxygen levels indicate a medical condition different from either of the two in isolation. Relying solely on practitioner experience presents its own set of problems because mental fatigue and poor acuity can lead to improper diagnoses and treatment.

Factors such as the ones described here, and other similar considerations, serve as obstacles to practitioners who attempt to gain a holistic picture; important cues that are missed result in incomplete or incorrect health assessments, leading to medical errors or deaths. In today's ecosystem, there is virtually no straightforward method for timely delivery of medical device data or for an integrated or holistic view of a patient's medical condition to practitioners not present at the patient's bedside.

Approximately one in three of the 13.75 million patients admitted to a hospital in the U.S. will experience an adverse event (AE), defined as “an unintended injury caused by medical management rather than the disease process” (e.g. falls with injuries, IV infiltration, hospital acquired infections, venous thrombosis, etc.). These adverse events contribute to approximately 400,000 deaths per year in hospitals at a cost of $100B. A substantial proportion of these adverse events are believed to be preventable because unusual oscillations in a patient's vital signs observed hours or days before an AE have been consistently identified as harbingers of an adverse event. These vital sign abnormalities often go undetected by healthcare providers in the hospital and precede AEs that result in a transfer to an intensive care unit and increased mortality.

Hospitals have attempted, with limited success, to reduce severe AEs by deploying early warning systems (EWS) or rapid response teams (RRTs) that respond to established predictors of AEs as they occur. However, a recent Scopus review concluded low level quantitative evidence exists that EWS does not reduce the incidence of severe adverse events. Findings of an earlier systematic review stated, “robust evidence to support RRT effectiveness in reducing hospital mortality is lacking.”

There are a variety of reasons why implementing EWS or ERTs have limited effect to prevent AEs. First, these systems, when employed outside of intensive care environments, do not provide real time data. Thus, there exists delays between the patient physiologically deteriorating, a practitioner assessing the deterioration, and interventions being introduced to prevent or treat the AE. Second, some of these systems employ population-based norms to determine patient deviations and not patient's specific variations in physiological functioning; using the former introduces broad parameters that are not sensitive to individual variations (e.g. age, gender, medications or disease state). Finally, EWS are dependent upon the validity and frequency of the data collected and the competency and availability of the individual interpreting the data.

A real non-medical issue that plagues the nursing staff in hospitals is fatigue, specifically alarm fatigue. In most hospital settings, nurses rely on hearing an alarm from a medical instrument and identifying the location of the alarm. Practitioners do not have a way to discern why an alarm is tripped other than entering the room and manually turning it off, and many alarms are caused by innocuous problems. Even when the alarm is the result of an issue, practitioners do not have the ability to determine the nature or severity of an alarm or its relationship to a patient's health without being physically present by the patient bedside. When there are multiple alarms sounding, the medical practitioner must, without context, attend to each alarm without knowing which alarm may need priority.

Technologies that have been proposed to communicate the presence of alarms using a communication network have done little to mitigate the problem, and instead, have created alarm fatigue among practitioners. Finally, the analytical tools provided by device manufacturers do not provide practitioners with any means of automating their Early Warning System protocols to help mitigate future problems. Instrument-based analyses are typically used simply to ensure that the instruments are functioning correctly.

To summarize, within non-ICU wards, the main drawback of Early Warning Systems is that they are reactive rather than proactive because they don't have access to real time data and are contingent upon population-based rules. The main drawback of EMRs is that they are difficult to use, also don't provide real-time data, and essentially function as a method to keep track of what a patient needs to be billed for; as mentioned in the exposition of the patent, EMRs get a “data-dump” from the vendor-specific servers roughly once or twice a day because the hospital network technologically cannot support hundreds of medical instruments all continuously pushing data—that would overload the network pretty quickly.

Thus, there is a need for a continuous, real-time monitor and patient-specific analytics system to help practitioners monitor patients remotely. There is a need for a device, system and method that can communicate medical data without giving practitioners alarm fatigue. There is a need for an improved EMR/EWS. There is also a need for a device, system and method which can push one cohesive image of a patient's progression and potential onset of adverse events to a mobile device in real-time.

### SUMMARY

The present disclosure, in its many embodiments, alleviates to a great extent the disadvantages of real-time remote access systems for medical data. Disclosed embodiments provide the solution to the overall problem, and the mobile application/patient proxy is the visual means in which this is realized. The patient proxy displays information from medical devices (agnostic to the vendor) in real time, is intuitive to use (designed with the help of the end user—nurses) and will incorporate patient-specific analytics to better predict trends and possible adverse events.

The present disclosure comprises patient proxies and methods of generating and presenting patient proxies. Disclosed proxies, systems, and methods integrate data from any medical instrument that is relevant to the development of a patient proxy in a timely manner or, as needed, in real time. Patient proxy systems and methods include means of acquiring data directly from medical instruments around a patient bedside, aggregating the data thus acquired to develop a short-term profile of a patient's medical condition, analyzing the data to ascertain the potential of certain patient-specific adverse events, and delivering both the data and the profile to a medical practitioner in a manner that is easily used and manipulated. Exemplary patient proxies can be thought of as an improved combination of an EMR and Early Warning Systems. The patient proxy does not intend to replace nurses or nurse judgement but rather acts as a supplementary resource to understand patient health.

In exemplary embodiments, delivery of the information and analysis will be to a smartphone, tablet or other mobile device that may be available to a medical practitioner. The information and analyses presented to the medical practitioner is a patient proxy, implying that disclosed systems and methods present the practitioner a proxy of the patient. Having a viewable patient proxy in real time on a mobile screen alleviates the need for practitioners to independently examine instruments on a room-to-room basis, which would not only save an immense amount of time, but it also provides practitioners with an entirely new framework of understanding patient health. With the patient proxy, the practitioner sees all the patient information in real-time without being confined to one location, essentially acting as an improved, real-time early warning system.

Exemplary patient proxies would also alleviate a significant amount of the stress caused by alarm fatigue; by having ubiquitous access to a patient's bedside treatment and medical devices and being able to cross reference medical device data with vital sign information through the patient proxy, practitioners would be able to understand and prioritize alarms by severity. Exemplary patient proxies would also aid practitioners in gauging potential adverse events based on a patient's current trends in data; juxtaposing patient-specific measured data with recorded cases of certain adverse events would help medical practitioners understand the different adverse events that could occur.

Exemplary embodiments of a computer-implemented patient proxy comprise data relating to a patient outputted by one or more medical instruments, one or more boundary parameters for the data, a data analytics system, repositories of existing symptoms of adverse events, algorithms to compare data, a communications system, and an alarm. In exemplary embodiments, the medical instruments include one or more of: infusion pumps, vital signs monitors, and bed scales.

The data analytics system determines which data comes from a particular medical instrument (i.e. metric), allows practitioners to set boundary conditions for each metric, determine whether the metric is outside the boundary parameters, and create a 99% confidence interval (CI) for each metric, and determines whether a patient may experience an adverse event through comparisons between the patient's progression and trends and an adverse event repository. The repository for adverse events consists of an electronic database of potential symptoms a patient may exhibit prior to the onset of an adverse event. The algorithms compare incoming device data with the 99% CI of each metric, create a snapshot of the patient's health based on the available data, and correlate the profile with the repository to determine which adverse events may occur.

The communications system communicates information about a patient's medical condition based on data determinations of the data analytics system and algorithms. The data may be outputted and communicated in real time. The alarm sounds when selected data is outside boundary parameters or a particular medical instrument has stopped functioning or changed its rate or if a patient may experience an adverse event. In exemplary embodiments, the alarm comprises a plurality of prioritizable alarms. The data analytics system may cross-reference data from one or more vital signs monitors with data from other medical instruments.

One of the key components of disclosed patient proxies is how the information is presented on a mobile device. In exemplary embodiments, the computer-implemented patient proxy comprises a user interface viewable on a mobile device. The user interface typically has at least one home screen displaying room numbers or bed numbers corresponding to patients but no patient information. In addition, the user interface may have at least one in-depth screen displaying patient metrics. In exemplary embodiments, the user interface also has at least one graphing screen displaying a patient's progression relating to a metric. The in-depth screen and/or the graphing screen may display data relating to any of the treatment devices and trends that are developing in the patient's health.

Exemplary computer-implemented methods of generating and presenting a patient proxy are also provided. Exemplary methods comprise aggregating data relating to a patient outputted by one or more medical instruments, creating one or more boundary parameters for the data, determining which data comes from a particular medical instrument and determining whether any data is outside the boundary parameters, comparing trends to existing adverse event symptoms, comparing data to symptoms of potential adverse events, communicating information about a patient's medical condition based on the data determinations, and sounding an alarm when selected data is outside pre-determined boundary parameters or a particular medical instrument has stopped functioning or changed its rate or if a patient may experience an adverse event. It should be noted that “sounding” an alarm could include a visual alarm or a vibration of some kind. A plurality of prioritizable alarms may be provided.

In exemplary computer-implemented methods, the one or more medical instruments include one or more of: infusion pumps, vital signs monitors, and bed scales. The determining step may include cross-referencing data from one or more vital signs monitors with data from other medical instruments. Exemplary computer-implemented methods may further comprise presenting the data and patient status via a user interface. In exemplary methods, room numbers or bed numbers corresponding to patients are displayed, as well as patient metrics, and patient's progression relating to metrics. Exemplary computer-implemented methods further comprise displaying data relating to an infusion pump. A color scheme may be presented wherein different colors represent how a patient is progressing.

Several variations exist, e.g., with different medical instruments; any combination of medical instruments can be used to create a patient proxy, so long as the combination is intended to create a way for practitioners to understand how the state of their patient evolves in real time. The figures and discussion herein show possible examples of a potential patient proxy that would be shown on a mobile device. The colors presented are based on a set of nurse input, and they believed that a green, yellow, and red color scheme was easiest to understand.

Exemplary embodiments can be divided into three distinct pieces: the treatment and bedside medical devices, the communication device or Care-Life Unit (CLU) and its embedded software, and the mobile application that presents the information. The application programming interface (API) may be a combination of technologies, including cloud functions, document-based data storage, remote device configuration, and push notifications. The API is the backbone of the solution infrastructure responsible for collating and disseminating bi-directional data. Ingress and egress data flow will occur in real-time. That is to say, that information written, applied, or committed to an API resource is immediately available to relevant components within the ecosystem. Furthermore, the API will centralize management of inventory data including, facilities, stations, and medical instruments and their corresponding default thresholds. The API will automate critical push notifications to alert end-users of threshold breaches.

The cross-platform mobile application will target current operating system versions on both iOS and Android and will provide nurses access to recorded patient vitals with limited administrative management of platform components. In the mobile application, a database module may be integrated with the API to handle document subscription, access to remote configuration, and anonymous authentication. A complete back-office collaboration solution will be provided with enhanced security, auditing capabilities, file storage, back-up solutions, and the remote management of mobile devices. Administrators will manage facilities and stations from the administrative portal.

The web-based administrative portal will provide an interface to administrators to manage the facility inventory, bed inventory, device inventory, and default configurations. Through the portal, administrators can manage locations, station and medical instrument inventory and default configuration values. This component could provide real-time reporting and surface other pertinent information to a dashboard-style interface summarizing the wider ecosystem status and usage. Administrators manage instrument inventory from the administrative portal. Instrument inventory may be saved as a remote configuration object identified by the parameter key instrument. In exemplary embodiments, this will be an array of instrument objects where each object contains the instrument name and default lower and upper bounds.

Infrastructure management will be provided to allow for remote device management to ensure security and operational usage integrity. Some of the pages for infrastructure management include facility, station, and instrument inventory management and station to facility relationships. Facility is an interface for adding and updating facilities and managing station association within the ecosystem. Facilities are recorded in the/facility resource and represent station allocation to a facility. Station is an interface for adding, updating and removing stations. Stations can be allocated to facilities from the facility management interface and represent all possible stations available within the associated facility. Stations are recorded in the/station resource and represent every room and bed combination. Because, as discussed in more detail herein, the patient proxy identifies rooms and beds in facilities, ecosystem complexity and size can be reduced by cataloging these only once as stations and then associating them with one or many facilities.

In exemplary embodiments, the device inventory is quite small. Therefore, as mentioned previously, remote configuration can be leveraged to inventory these items as an array of instrument objects. These will be accessible and manageable within the administrative portal whereby new instruments can be added and existing instruments can be modified. This feature will also provide the interface for setting an instrument's lower and upper bounds. Remote configuration will maintain the device inventory and provide a means to initialize configured profiles with default lower and upper bounds as well as any other necessary application-specific configuration data. Authenticating into the administrative portal will be based on the user managed within the admin interface. Access control may evolve based on the organizational units' roles managed within the cloud console.

It is an object of disclosed embodiments to wirelessly acquire and analyze data from three treatment and monitoring devices (i.e. Infusion Pumps, Vital Signs Monitors, and Bed Scales) and communicate a cohesive patient profile with a handheld device through the mobile application. It is an object of disclosed embodiments to wirelessly acquire and analyze data from treatment and monitoring devices attached to a hospitalized patient and communicate changes in the patient's condition to a nurse's handheld device in a live hospital environment. It is an object of disclosed embodiments to wirelessly connect to the three bedside instruments, analyze incoming data, and push all relevant patient data and potential onset adverse event information to the healthcare practitioner in both simulated and live environments.

Accordingly, it is seen that patient proxies, systems, and methods of generating and presenting patient proxies are provided. These and other features and advantages will be appreciated from review of the following detailed description, along with the accompanying figures in which like reference numbers refer to like parts throughout.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1a is a schematic of an exemplary embodiment of a patient proxy system in accordance with the present disclosure;

FIG. 1b is a schematic of an exemplary embodiment of a patient proxy system in accordance with the present disclosure;

FIG. 2a is a process flow diagram of an exemplary embodiment of a method of generating a patient proxy showing data analytics processes in accordance with the present disclosure;

FIG. 2b is a process flow diagram of an exemplary embodiment of a method of generating a patient proxy showing data analytics processes in accordance with the present disclosure;

FIG. 2c is a process flow diagram of an exemplary embodiment of a method of generating a patient proxy showing data analytics processes in accordance with the present disclosure;

FIG. 3 is a schematic of an exemplary embodiment of a user interface showing an exemplary home screen in accordance with the present disclosure;

FIG. 4 is a schematic of an exemplary embodiment of a user interface showing an exemplary home screen in accordance with the present disclosure;

FIG. 5 is a schematic of an exemplary embodiment of a patient proxy showing an exemplary in-depth screen in accordance with the present disclosure; and

FIG. 6 is a schematic of an exemplary embodiment of a user interface showing an exemplary graphing screen in accordance with the present disclosure.

### DETAILED DESCRIPTION

In the following detailed description of exemplary embodiments of the disclosure, reference is made to the accompanying drawings in which like references indicate similar elements, and in which is shown by way of illustration specific embodiments in which disclosed systems and devices may be practiced. These embodiments are described in sufficient detail to enable those skilled in the art to practice the embodiments, and it is to be understood that other embodiments may be utilized and that logical, mechanical, functional, and other changes may be made without departing from the scope of the present disclosure. The following detailed description is, therefore, not to be taken in a limiting sense, and the scope of the present disclosure is defined by the appended claims. As used in the present disclosure, the term “or” shall be understood to be defined as a logical disjunction and shall not indicate an exclusive disjunction.

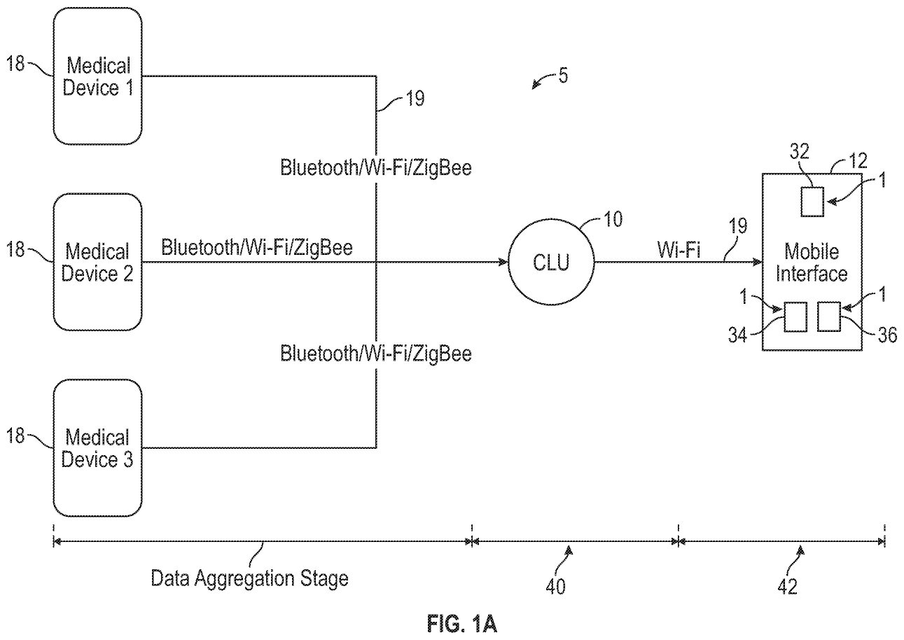

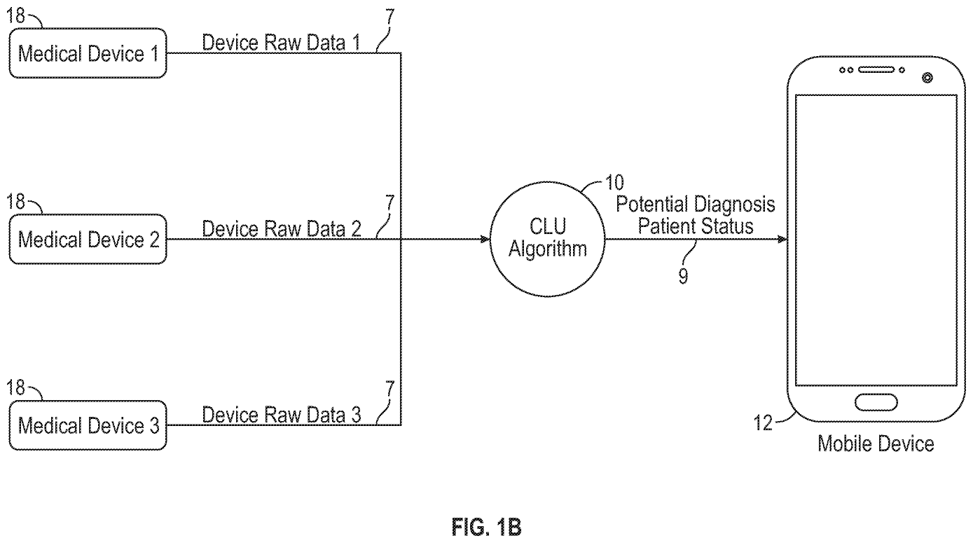

FIG. 1a is a high-level schematic of exemplary system architecture that could provide a patient proxy system 5. Disclosed embodiments comprise hardware, software and firmware. More particularly, a communication device 10, as described in detail in co-pending U.S. patent application Ser. No. 15/962,290, filed Apr. 25, 2018, and hereby incorporated by reference in its entirety, has a transceiver (not shown) and circuit (not shown) having a communication channel. The device 10 has a processor (not shown) and memory (not shown) in communication with the transceiver circuit. Typically, embodiments will have one transceiver circuit including one transceiver, but some embodiments may have a second transceiver circuit. The device 10 receives medical data from at least one medical instrument 18, which may be equipped with a transceiver or dongle. The medical instrument could be an infusion pump, or any other medical instrument that monitors or provides medication or treatment to patients. In the current hospital environment, medical devices (such as infusion pumps) take pertinent patient readings and measurements. In exemplary embodiments, there are three medical instruments or types of instruments 18 (Infusion Pumps, Vital Signs Monitors, and Bed Scales). Of course, there are other bedside treatment and medical devices that can be integrated in the future.

This information may be sent over a local area network (LAN) 19 to an on-site server (not shown). The server, while controlling most of the software related to medical devices sends the data over Wi-Fi to various electronic medical record systems (EMRs). In exemplary embodiments, the communication device 10 transmits the medical data to a medical practitioner's mobile device 12. More particularly, and as described in detail herein, the communication device 10 is able to pull data from medical instruments, coordinate and analyze that information before sending it to its final destination. As discussed in more detail herein, there are three components to building the patient proxy: data aggregation, data coordination and analysis, and data presentation. Disclosed embodiments may use Wi-Fi, Bluetooth, ZigBee, and/or other wireless communication methods.

FIG. 1b is a high-level schematic of an exemplary patient proxy system 5 illustrating general data flow. The raw data 7 from each medical instrument 18 is transmitted to the communication device 10. The medical instrument could be an infusion pump, or any other medical instrument that monitors or provides medication or treatment to patients. The communication device performs the data coordination and analysis described in more detail herein and then passes the resulting information, including possible potential diagnoses and patient status 9, to the medical practitioner's mobile device 12.

In exemplary methods of generating and presenting a patient proxy, there are three components or steps to building the patient proxy: data aggregation, data coordination and analysis, and data presentation. Data aggregation is the first step in creating the patient proxy; it consists of capturing data outputted by any number of medical instruments in real time via any form of device communications (e.g. Wi-Fi, Bluetooth, ZigBee, etc.). Medical instruments are typically designed to output information to the EMRs and need to be redirected towards the CLU; redirection can be done via hardware or software.

The second stage in creating the patient proxy is the data coordination and analysis stage, performed by a data analytics system 40. The first feature of the second stage is parsing through all the information that comes into the communication device 10 and determining which information belongs to which medical instrument 18. Practitioners will also be able to input boundary parameters for any of the statistics that are measured; when any of the measured data do not fit inside the boundary conditions, the nurse will be alerted based on what the software has found. Exemplary embodiments of the software will be able to cross reference medical device data and alarms with vital signs monitor information to give practitioners a better understanding of how their medical devices are affecting the patient in real time.

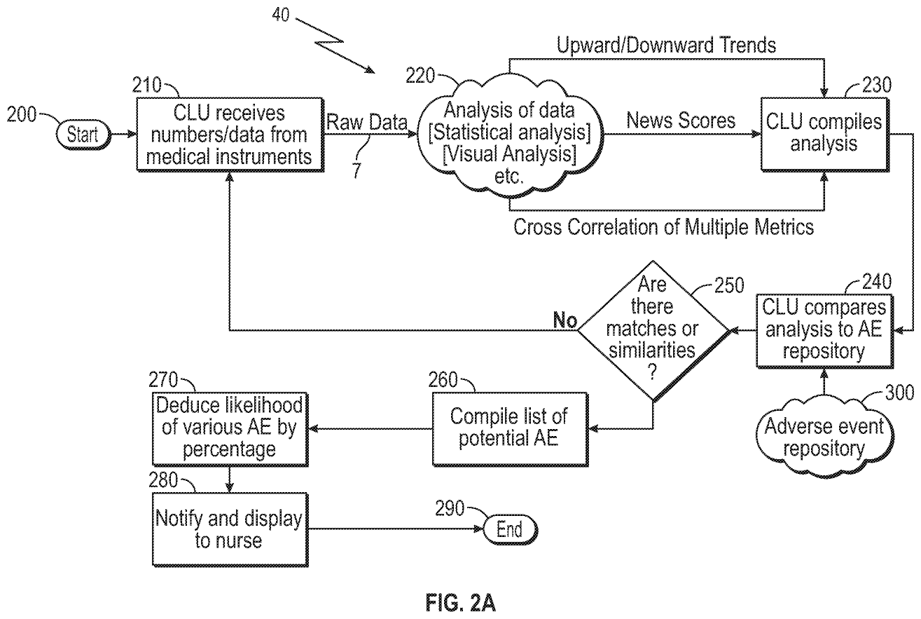

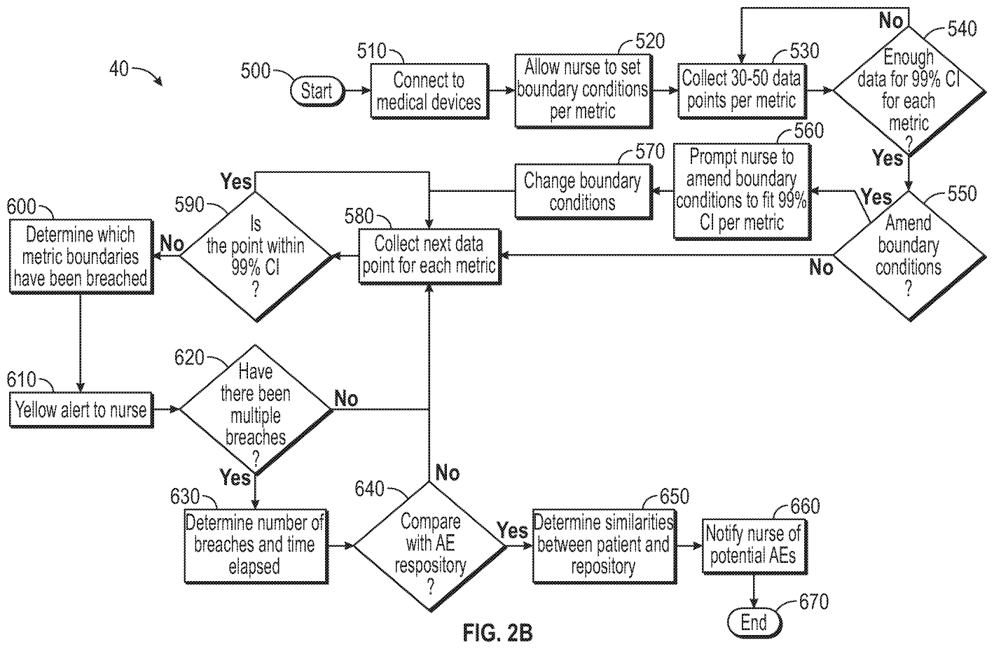

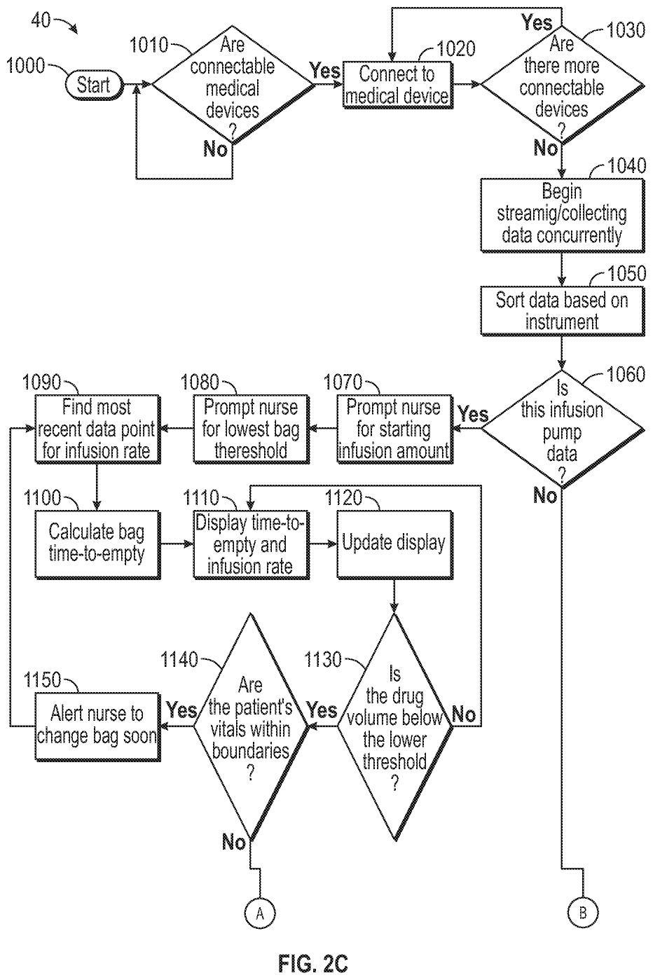

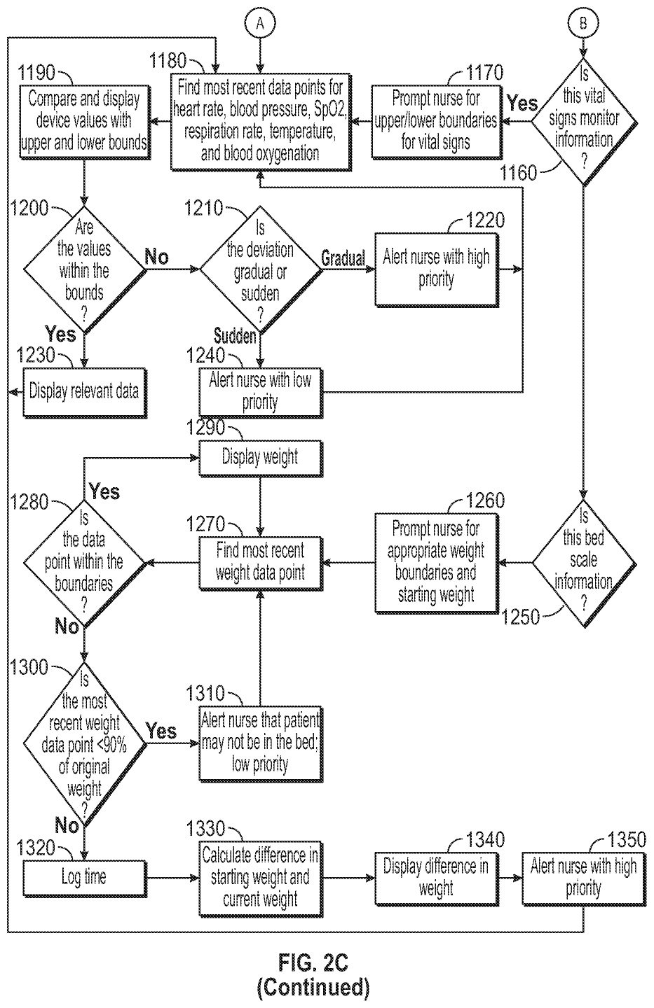

With reference to FIGS. 2a, 2b, and 2c process flows for exemplary Data Coordination and Analysis stages, performed by data analytics system 40, for the patient proxy will now be described. There are (at least) two pieces that go into the data analytics facet of the patient proxy: the repository of AE symptoms and the algorithms that both disseminate trends (i.e. physiological symptoms) from the measured bedside data and compare 240 said symptoms to the repository. The AE repository 300 serves as the “master-guide” for the different AE that a patient may exhibit; events such as sepsis, infection, and cardiac arrest may be predicted by variations in independent vitals and through the cross examination of multiple vitals together. The repository can be comprised of existing literature (e.g. case studies) and examined data retroactively gathered from AE that occur in the hospital. The microanalysis algorithms essentially function as a means to which raw patient data, measured from the various bedside medical instruments, can be converted into trends that are compared to the AE repository; an example of this is the transformation of three hours of measured pulse rate data to a potential directional trend and/or comparison to the transformation of another measured metric. The microanalysis algorithms will also then be able to compare the deduced trends to the AE repository, ascertain the potential of certain AEs, and notify the responsible practitioners.

More particularly, as illustrated in FIG. 2a, at the start 200, the communication device receives 210 numbers and/or data from the medical instruments. That raw data 7 is analyzed 220, including, e.g., upward/downward trends, MEWS scores, and cross-correlation of multiple metrics, and the communication device compiles 230 its analysis. The communication device compares 240 its analysis to the information in the AE repository 300 and queries 250 whether there are matches or similarities. Then the system compiles 260 a list of potential adverse events, deduces 270 the likelihood of various AEs by percentage, and notifies and displays 280 that information to the nurse or medical practitioner, and the process ends 290.

As shown in FIG. 2b, an exemplary process starts 500 and connects to medical devices 510. The nurse or other medical practitioner is allowed to set boundary conditions per metric 520. In exemplary embodiments, the data analytics system 40 collects 30-50 data points per metric 530 and then queries 540 whether that is enough data for 99% CI for each metric. If not, the system repeats the collection step 530 and collects another 30-50 data points per metric. If that is enough data, the system queries whether the boundary conditions need to be amended 550. If the answer is yes, the nurse is prompted to amend the boundary conditions to fit 99% CI per metric 560 and then the boundary conditions are changed 570. If the boundary conditions do not need to be amended, the next data point for each metric is collected 580. The data analytics system 40 queries whether the point is within 99% CI 590. If not, the system determines which metric boundaries have been breached 600 and sends 610 a yellow alert to the nurse or medical practitioner. The system then queries 620 whether there have been multiple breaches and, if so, determines 630 the number of breaches and time elapsed. That data is compared 640 with the AE repository and the system determines similarities between the patient and repository 650 and notifies the nurse of potential AEs 660. In exemplary embodiments, the data analytics flow ends 670 at that point.

Turning to an exemplary Data Coordination and Analysis stage in FIG. 2c, after starting 1000, exemplary patient proxy methodology seeks any connectable medical devices 1010 in the network and connects with such devices 1020 if there are such devices. The patient proxy may look for more connectable devices 1030 and, if there are no others, will begin streaming/collecting data concurrently 1040 from the devices. The data is sorted 1050 based on which medical instrument it is from. In exemplary embodiments, the patient proxy queries 1060 whether the data is from an infusion pump 1060, is vital signs monitor information 1160, or bed scale information 1250. Depending on which of these instruments the data is from, a particular process flow is triggered.

If the data is from an infusion pump 1060, the patient proxy may prompt the nurse or other medical practitioner for the starting infusion amount 1070 and prompt the nurse for the lowest bag threshold 1080. Then the patient proxy finds the most recent data point for the infusion rate 1090 and calculates the bag time-to-empty 1100. It then displays the time-to-empty and infusion rate 1110 and updates the display 1120 on the practitioner's mobile device. The patient proxy then queries 1130 whether the drug volume is below the lower threshold or boundary. If not, it again displays the time-to-empty and the infusion rate 1110. If the drug volume is below the lower threshold, the patient proxy queries 1140 whether the patient's vitals are within the boundaries. If the patient's vitals are within the boundaries, the patient proxy alerts the nurse to change the infusion bag soon 1150 and again finds the most recent data point for the infusion rate 1090. If the patient's vitals are not within the boundaries, the patient proxy may perform a query from the vital signs monitor information process flow such as finding the most recent data points for patient vitals 1180.

If the data is from a vital signs monitor 1160, the patient proxy prompts the nurse or other medical practitioner for the upper and lower boundaries for the vital signs 1170. The patient proxy finds the most recent data points 1180 for such vitals as heart rate, blood pressure, SpO2, respiration rate, temperature, and blood oxygenation. The patient proxy then compares and displays the device values with the upper and lower bounds 1190 and queries 1200 whether the values are within the bounds. If they are, the relevant data are displayed 1230. If the values are not within the bounds, it queries 1210 whether the deviation is gradual or sudden. If sudden, the patient proxy alerts the nurse with high priority 1240; if gradual, it alerts the nurse with low priority 1220.

If the data is bed scale information 1250, the patient proxy prompts the nurse for the appropriate weight boundaries and starting weight 1260. The patient proxy finds the most recent weight data point 1270 and queries whether the data point is within the boundaries 1280. If it is, it displays the weight 1290. If the data point is not within the boundaries, the patient proxy queries 1300 whether the most recent data point is less than 90% of the patient's original weight. If it is, the patient proxy alerts the nurse that the patient may not be in the bed 1310; this is a high priority alert. If the most recent data point is not less than 90% of the patient's original weight, the time is logged 1320, the difference between the starting weight and the current weight is calculated 1330 and displayed 1340, and the nurse alerted with low priority 1350.

As suggested by the data coordination and analysis described above, one important difference between the patient proxy and individual device analysis is that non-vital signs monitoring machines will be cross analyzed with vital signs information. In this way, a practitioner now understands how each medical device impacts the overall health and status of the patient through vital signs information in real time.

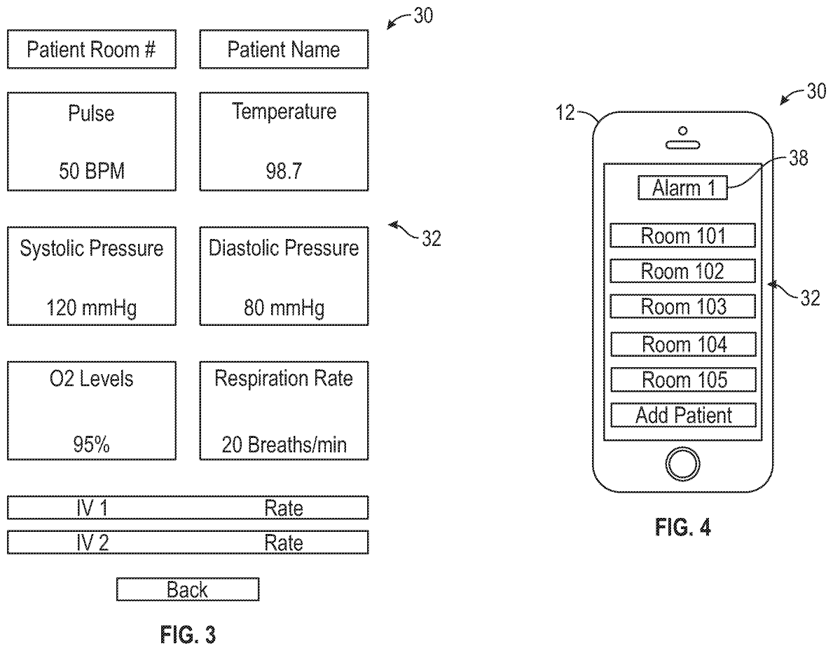

The third stage in generating a patient proxy is how the information is presented on a mobile device by a communications system 42. In exemplary embodiments, communications system 42 has several screens detailing the patient proxy. Turning to FIGS. 3-6, exemplary embodiments of a patient proxy will now be described. A patient proxy 1 is a cohesive image of a patient's medical information and progression, which is pushed to a mobile device 12 in real-time. Exemplary embodiments do that through a software application on mobile devices, which, in exemplary embodiments has three layers to it: Home Screens 32, In-Depth Screens 34, and Graphing Screens 36. It should be noted that additional layers and screens could be added depending on the application. These three screens 32, 34, 36 together are intended to provide the healthcare practitioner a way to understand how his/her patients are recovering without the need to manually inspect the instruments, which if a practitioner has multiple patients, can be incredibly difficult to do. Although a patient proxy is a virtual construct, for purposes of illustration, it can be thought of as the information presented in the three screens, which together are represented in FIG. 1 by reference number 1.

FIGS. 3-6 show an exemplary graphical or user interface 30 for the patient proxy. In exemplary embodiments, the user interface 30 displays the history of the patient's progression based on a single metric, which could be the pulse. As it pertains to the patient proxy, the user/graphical interface 30 gives practitioners the ability to see trends in the data, as well as, artifacts. The former is helpful in the event that a patient's metric begins to move in a certain direction, the graphical interface will alert the practitioner that he/she is potentially experiencing the onset of an adverse event. The latter pertains to artifacts in data; this refers to the idea that sometimes alarms are caused by “trips” or hiccups in the medical device (whether it be the connection to the human body or on the machine itself). In either case, an alarm sounds when it detects something to be awry. The graphical interface 30 gives the practitioner the ability to see if the reason a metric rose above or fell below the threshold was a “sudden spike” (which typically refers to a malfunction) or an actual trend in which the patient may be suffering from an adverse event.

FIGS. 3 and 4 show exemplary embodiments of a home screen 32 for the mobile application. The purpose of this screen is to give the healthcare practitioner a quick method to ascertain the status of his or her patients; the first “home” page of a patient proxy is a way for practitioners to quickly see how their patient is doing. In exemplary embodiments, to protect patient information and safety, the information shown could be by room number or bed number. Each room number or bed number corresponds to a specific patient. The icons on the right, e.g., drip, heart rate, pulse, are designed to give the practitioner an extra level of detail on the home page, and each icon is tied to a measured metric from the medical devices that the patient is connected to; in this way, the practitioner can gauge the recovery of each patient and understand what potential adverse events may arise.

In exemplary embodiments, a color scheme is employed where each color represents how the patient is progressing. The colors are determined by a combined consensus of data from healthcare practitioner knowledge, medical device data, and the app itself. There is no limit on the number of colors or what is considered a green, yellow, and red. In exemplary embodiments, green refers to a patient who is not exhibiting any potential onset of adverse events; it indicates that the patient is progressing smoothly. Yellow could refer to a patient who may be experiencing discomfort and should be attended to when possible; it indicates that the patient has encountered some error but may not require immediate attention (would trigger a low priority alarm 38). Red refers to a patient who is experiencing an adverse event and needs to be attended to immediately; it indicates that the patient has experienced an error that requires immediate attention (would trigger a high priority alarm 38).

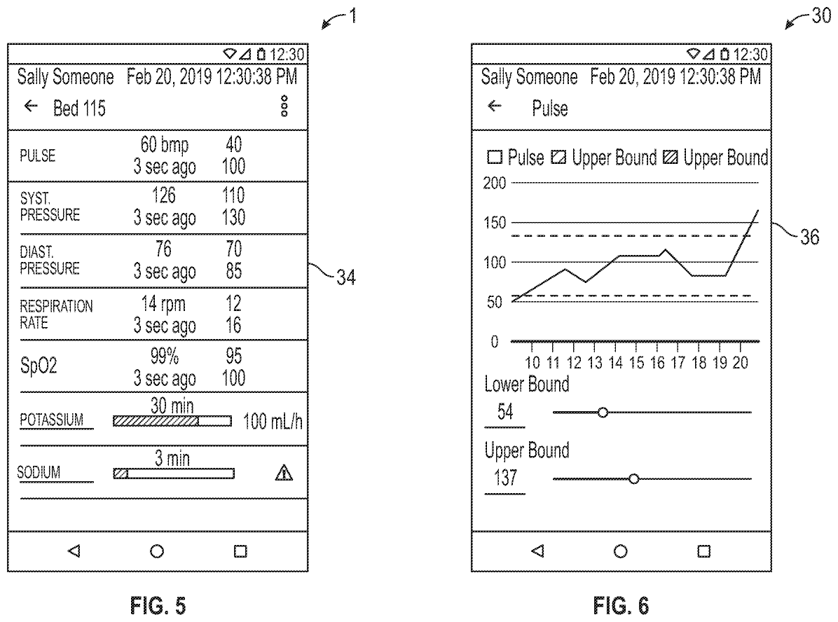

Turning to FIG. 5, an exemplary in-depth screen or page 34 will be described. An exemplary in-depth page 34 shown in FIG. 5 might appear when a medical practitioner selects any of the rooms or beds displayed on a home screen 32. Here, a practitioner would be able to see how all the medical instruments are operating, enter various boundary conditions, and ensure that the patient is within those conditions. As discussed above, color-coding may be utilized, and the yellow triangle here is an example of the system alerting a medical practitioner that the patient may need medical assistance in the future, which in this case, is a near empty infusion bag. Instead of responding to an already empty bag, the practitioner can track the bag's status on his/her phone and preemptively address the situation before it occurs.

The in-depth page 34 also gives a tremendous amount of assistance relating to the onset of potential adverse events. Fluctuations in independent vital signs have consistently been identified as harbingers of potential adverse events. However, a substantial number of these adverse events are undetected because there is no one there to observe the changes in these readings; with the in-depth page, a practitioner now has the ability to do so.

FIG. 6 is an example of a patient's progression provided by graphing screen 36. Each metric shown on the application is measured from the treatment and medical devices connected to the patient (in this case Infusion Pumps, Vital Signs Monitors, and Bed Scales) and may be transferred to a mobile interface via the communication device 10 and/or the cloud. In exemplary embodiments, on each metric there are numbers on the right that act as soft boundaries for the metric measured. For example, if one looks at Pulse, the application is set to alert the main healthcare practitioner when the pulse drops below 40 beats per minute or exceeds 100 beats per minute. Each measurement may be time stamped to help practitioners understand when the metric data came in.

In operation, in addition to presenting information from various medical devices as described above, exemplary patient proxies, systems and methods also feature the ability to input upper and lower boundaries for any of the medical device parameters that are being collected or displayed. This serves as the practitioner's ability to prioritize alarms and reduce alarm fatigue. This can span across multiple instruments as well, and an example of this would be as follows: (1) a nurse wishes to be alarmed when an infusion has less than 5 minutes of fluid left, when the infusion rate changes unexpectedly or stops, or if the vitals deviate from set values; (2) a patient is resting and accidentally sleeps on the IV line, causing the pressure to build in the line and forcing the IV machine to stop infusing; (3) the rate on the machine has now changed (or stopped) causing an alarm to sound by the pump; (4) patient's vitals are not changing outside of the set boundaries.

Such alarms and/or push notifications may be provided by cloud messaging. In exemplary embodiments, push notifications serve two purposes. The first is to engage nurses with real-time document model updates. The second will allow a conduit for real-time messaging to end-users; this could be uni- or bi-directional. This aspect of the complete solution can allow for live chat or some other messaging solution.

In exemplary embodiments, cloud functions will provide a minimalistic interface for detecting changes to document models where mobile devices are not actively subscribed. This will resolve the inactive nature of mobile applications in background mode, which pause subscriptions to document models. These subscriptions will resume when the application once again comes to the foreground. A push notification is dispatched when a breach occurs in the lower or upper bound thresholds prompting the user to bring the application into the foreground where the existing subscriptions will be re-engaged.

In today's ecosystem with an alarm now sounding, a nurse would have to enter the room to turn off the alarm and assess the situation without knowing that the problem is a patient sleeping on the IV line. Unfortunately, practitioners will only hear that a device is malfunctioning and nothing more, and there are often many alarms sounding at once. Since alarms do not convey severity, a practitioner must choose whether to answer an alarm now or later. However, with the patient proxy, the nurse can look at his or her mobile device and see that none of the patient's vitals are changing even if the infusion rate has stopped. Because the vital signs are still within the set boundaries, the practitioner can see that this alarm does not necessarily need immediate attention, and if there is a more urgent problem, then the practitioner can tend to that problem first.

In exemplary embodiments, a patient proxy will employ anonymous authentication which means it is not required for each user to have a Google account or even an account based on a username and password combination, PIN number, or phone number. End-user devices, however, may be protected by a remotely managed authentication protocol which may be a screen lock PIN or password. The primary application features will function by subscribing to the/profile API resource where updates provided from the communication device are immediately available to end-user devices. End-user devices must first be initialized by authentication with a service account and attached to admin device management so device policy can be remotely enforced before being delivered to a facility for use. Admin device management will enforce security and application restrictions for devices within the ecosystem and network settings on individual devices.

A profile is a collection of data binding a device to a station and a station to instruments. A profile is defined by the collection of a station, a mobile device, a communication device, and any connected medical instruments. Profiles are recorded in the/profile resource and represent the binding of a device to a station and associated instruments. The end-user device application will scaffold the new/profile document at the API based on the remote configuration blueprint. Configuring a profile involves two steps: tethering and confirmation.

Tethering involves connecting to a communication device and associating nearby medical instruments. All non-tethered devices will intermittently broadcast its connectivity state for the end-user to identify and initiate a connection. Once connected the communication device will temporarily connect to all nearby non-tethered Bluetooth-attached medical instruments and provide feedback through the API of its readings to aid in the identification of the correct instrument. The end-user will be presented with a screen to confirm the configuration is correct. If a mistake was made during tethering the process will begin again. If the configuration is correct the communication device will drop connections with the remaining instruments and report the final attached instrument inventory to the API and reinitialize the chart.

It should be noted that exemplary proxies and methods do not give or make treatment decisions. They simply act as a data aggregator and presenter, so the medical practitioner does not need to take the steps to find the bits and pieces of information for him/herself.

While the disclosed systems and devices have been described in terms of what are presently considered to be the most practical exemplary embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

Thus, it is seen that improved communication devices, systems, and methods are provided. It should be understood that any of the foregoing configurations and specialized components may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

1. A computer-implemented patient proxy comprising:
data relating to a patient outputted by one or more medical instruments;
one or more boundary parameters for the data;
a data analytics system determining which data comes from a particular medical instrument, determining whether any data is outside the boundary parameters, and determining whether a patient may experience an adverse event through comparisons between the patient's progression and trends and an adverse event repository;
a communications system communicating information about a patient's medical condition based on data determinations of the data analytics system; and
an alarm sounding when selected data is outside pre-determined boundary parameters or a particular medical instrument has stopped functioning or changed its rate, or a patient may experience an adverse event.

2. The computer-implemented patient proxy of claim 1 wherein the data is outputted and communicated in real time.

3. The computer-implemented patient proxy of claim 1 wherein the one or more medical instruments include one or more of: infusion pumps, vital signs monitors, and bed scales.

4. The computer-implemented patient proxy of claim 3 wherein the data analytics system cross-references data from one or more vital signs monitors with data from other medical instruments.

5. The computer-implemented patient proxy of claim 1 further comprising a user interface viewable on a mobile device.

6. The computer-implemented patient proxy of claim 5 wherein the user interface has at least one home screen displaying room numbers or bed numbers corresponding to patients.

7. The computer-implemented patient proxy of claim 5 wherein the user interface has at least one in-depth screen displaying patient vital signs data or other measured metrics.

8. The computer-implemented patient proxy of claim 5 wherein the user interface has at least one graphing screen displaying a patient's progression relating to a vital sign or other measured metrics.

9. The computer-implemented patient proxy of claim 5 wherein the user interface has an in-depth screen displaying data relating to an infusion pump or other measured metrics.

10. The computer-implemented patient proxy of claim 1 wherein the alarm comprises a plurality of prioritizable alarms.

11. A computer-implemented method of generating and presenting a patient proxy, comprising:
aggregating data relating to a patient outputted by one or more medical instruments;
creating one or more boundary parameters for the data;
determining which data comes from a particular medical instrument and determining whether any data is outside the boundary parameters;
creating trends based on the aggregated data;
comparing trends to existing adverse event symptoms;
communicating information about a patient's medical condition based on the data determinations;
sounding an alarm when selected data is outside pre-determined boundary parameters or a particular medical instrument has stopped functioning, changed its rate, or if a patient may experience an adverse event.

12. The computer-implemented method of claim 11 wherein the one or more medical instruments include one or more of: infusion pumps, vital signs monitors, and bed scales.

13. The computer-implemented method of claim 12 wherein the determining step includes cross-referencing data from one or more vital signs monitors with data from other medical instruments.

14. The computer-implemented method of claim 11 further comprising presenting the data and patient information via a user interface.

15. The computer-implemented method of claim 14 further comprising displaying room numbers or bed numbers corresponding to patients.

16. The computer-implemented method of claim 14 further comprising displaying patient vital signs data or other measured metrics.

17. The computer-implemented method of claim 14 further comprising displaying a patient's progression relating to a vital sign.

18. The computer-implemented method of claim 14 further comprising displaying data relating to an infusion pump.

19. The computer-implemented method of claim 11 further comprising providing a plurality of prioritizable alarms.

20. The computer-implemented method of claim 11 further comprising presenting a color scheme wherein different colors represent how a patient is progressing.
